(19) United States
(12) Patent Application Publication
(10) Pub. No.: US 2025/0352831 A1
(43) Pub. Date: Nov. 20, 2025

(54) ULTRASOUND THERAPY TRANSDUCER FOR HISTOTRIPSY SYSTEMS AND METHODS

(71) Applicant: HistoSonics, Inc., Plymouth, MN (US)

(72) Inventors: Jonathan M. CANNATA, Ann Arbor, MI (US); Ryan M. MILLER, Saline, MI (US); Tyson DELANDSHEER, Whitmore Lake, MI (US); Daniel S. MARXER, Eagan, MN (US)

(21) Appl. No.: 19/210,971

(22) Filed: May 16, 2025

Related U.S. Application Data
(60) Provisional application No. 63/649,153, filed on May 17, 2024.
(60) Provisional application No. 63/700,125, filed on Sep. 27, 2024.

(51) Int. Cl. A61N 7/02; A61N 7/00; B06B 1/06

(52) U.S. Cl. CPC A61N 7/02; B06B 1/0625; B06B 1/067; A61N 2007/0039; A61N 2007/0082; B06B 2201/76

(57) ABSTRACT

A histotripsy therapy system configured for the treatment of tissue is provided, which may include any number of features. Provided herein are histotripsy and/or ultrasound transducer arrays, and associated systems and methods that provide efficacious non-invasive and minimally invasive therapeutic, diagnostic and research procedures. The transducer arrays provided herein can include a machined array shell having a plurality of wells formed therein. An acoustic stack including a plurality of matching and transducer elements can be disposed in the wells and be configured to transmit ultrasound energy through the plurality of matching layers and through the array shell towards a common focal point

### PRIORITY CLAIM

This patent application claims priority to U.S. provisional patent application No. 63/649,153, titled “ULTRASOUND THERAPY TRANSDUCER FOR HISTOTRIPSY SYSTEMS AND METHODS”, and filed on May 17, 2024, and U.S. provisional patent application No. 63/700,125, titled “ULTRASOUND THERAPY TRANSDUCER FOR HISTOTRIPSY SYSTEMS AND METHODS”, and filed on Sep. 27, 2024, which are all herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU), cryo, or radiation, Histotripsy relies on the mechanical action of cavitation for tissue destruction and not on heat, cold or ionizing energy.

Transducer design and manufacturing for Histotripsy capable transducers is incredibly difficult to achieve. Currently, Histotripsy transducers are manufactured by shaping their piezoelectric composite and matching layer to the desired radius of curvature for the transducer design. Bulk piezoelectric and matching layer materials are both manufactured flat. The piezoelectric material is cut in two orthogonal directions to form a plurality of diced posts in the material, which are then filled with epoxy resin. This curving process can be very challenging due to the stress that it imparts on the composite structure. The cured or semi-cured epoxy needs to widen to accommodate the curving as the piezoelectric posts/pillars are rigid. Dis-bonding of the epoxy and piezoelectric post/pillar or crack formation can occur leading to physical or electrical failure of the structure.

Additionally, transducer frequency is dictated by composite thickness, therefore the likelihood of these failures to occur increase with a decrease in transducer frequency for a given transducer radius of curvature. As a result, prior therapy transducers are limited in the driving frequencies they can support, since lower therapy frequencies require thinner Also, there are similar limitations with respect to the percentage of the composite that is occupied by piezoelectric material. This is termed piezoelectric composite volume fraction. With a higher piezoelectric volume fraction there is less epoxy filler to widen during the curving process.

### SUMMARY OF THE DISCLOSURE

A transducer array is provided, comprising: an array shell comprising a solid emitting surface and a rear surface, a plurality of wells formed in the rear surface; a plurality of first matching layers individually disposed in the plurality of wells and contacting the proximal surface; and a plurality of transducer elements disposed in the wells and contacting the plurality of first matching layers, the plurality of transducer elements being configured to transmit ultrasound energy through the plurality of first matching layers and through the solid emitting surface towards a common focal point.

In some aspects, the solid emitting surface forms a second matching layer for the plurality of transducer elements.

In one aspect, the solid emitting surface is concave.

In some aspects, the solid emitting surface has a contiguous smooth curvature.

In one aspect, the solid emitting surface has flat facets corresponding to each of the plurality of wells formed in the rear surface.

In some aspects, the plurality of wells have the same surface area.

In other aspects, the plurality of wells have varying shapes. In some aspects, the plurality of wells have varying shapes.

In one aspect, a bottom surface of each of the plurality of wells is flat.

In some aspects, each of the plurality of transducer elements are sized and shaped to fill out all edges of a corresponding well of the plurality of wells.

In another aspect, the plurality of wells are formed by a machining process.

In some aspects, the array shell comprises a plastic material.

In one aspect, the plurality of first matching layers comprises a polymer composite material.

In other aspects, the polymer composite material includes glass or ceramic particles disposed therein.

In additional aspects, the plurality of first matching layers has an acoustic impedance of about 5-8 Megarayl.

In another aspect, the plurality of first matching layers has a thickness of from about 0.5 mm to 1.5 mm.

In one aspect, each of the plurality of transducer elements includes an electrical connection interface.

In other aspects, each of the plurality of transducer elements includes a first notch or cutout configured to allow the electrical connection interface to receive one or more wires.

In additional aspects, each of the plurality of first matching layers comprises a second notch or cutout configured to align with the electrical connection interface and first notch or cutout of a corresponding transducer element of the plurality of transducer elements.

In another aspect, each of the plurality of transducer elements comprises a second notch or cutout configured to be used as a guide for applying an adhesive or encapsulating layer between each of the plurality of transducer elements and the plurality of first matching layers.

In one aspect, the electrical connection interface is disposed in a corner of each of the plurality of transducer elements.

In yet another aspect, a bottom surface of each of the plurality of wells has curved edges.

In some aspects, the plurality of first matching layers have curved front edges to match the curved edges of the plurality of wells.

In an additional aspect, the plurality of transducer elements comprise a piezoelectric-polymer composite material.

In some aspects, the plurality of transducer elements comprise a solid piezoelectric material.

In yet another aspect, the plurality of transducer elements comprise a silicon material formed with microelectromechanical systems (MEMS) technology.

In one aspect, the transducer array includes an overmolded epoxy layer disposed on the solid emitting surface of the array shell.

In another aspect, the overmolded layer forms a second matching layer.

In one aspect, the overmolded layer comprises an epoxy or urethane.

In another aspect, the transducer array comprises a central aperture disposed in the array shell configured to receive an ultrasound imaging probe.

A transducer array is provide, comprising: an array shell comprising a front emitting surface; a plurality of transducer elements disposed in a rear surface of the array shell and configured to transmit ultrasound energy through the front emitting surface towards a common focal point; at least one interconnect assembly facilitating a plurality of electrical connections from a signal generator to each of the plurality of transducer elements, the at least one interconnect assembly including one or more capacitors at each electrical connection configured to tune an operating parameter of the plurality of transducer elements to the signal generator.

In one aspect, the one or more capacitors are selected to tune the plurality of transducer elements to a desired operating frequency.

In another aspect, the one or more capacitors are selected to tune the plurality of transducer elements to achieve a desired operating frequency lower or higher than what is specified for the plurality of transducer elements based upon a thickness and material composition of the plurality of transducer elements.

In some aspects, the one or more capacitors are selected to achieve a desired acoustic pulse shape.

In another aspect, the one or more capacitors have a capacitance ranging from 1 to 1000 pF.

In some aspects, the one or more capacitors include more than one capacitance to produce a multi-frequency transducer array.

In additional aspects, the interconnect assembly comprises a printed circuit board (PCB).

In some aspects, the at least one interconnect assembly is disposed between a back cover of the transducer array and the array shell.

In another aspect, the one or more capacitors form a parallel connection between a positive connection and a ground connection to each transducer element.

In some aspects, the plurality of transducer elements have the same surface area.

In another aspect, the plurality transducer elements have varying shapes.

An ultrasound system is provided, comprising: a generator configured to generate histotripsy waveforms; a robotic positioning arm comprising a mechanical connection for physical attachment to a plurality of ultrasound treatment heads; an electrical connection for electrical coupling between the generator and the plurality of ultrasound treatment heads; a first ultrasound treatment head having a first focal length and comprising a first plurality of transducer elements each having a first surface area, the first plurality of transducer elements being electrically coupled to at least one capacitor configured to tune the first plurality of transducer elements to resonate at a desired operating frequency when the first ultrasound treatment head is electrically coupled to the generator; and a second ultrasound treatment head having a second focal length and comprising a second plurality of transducer elements each having a second surface area different than the first surface area of the first plurality of transducer elements, the second plurality of transducer elements being electrically coupled to at least one capacitor configured to tune the second plurality of transducer elements to resonate at the desired operating frequency when the second ultrasound treatment head is electrically coupled to the generator.

In one aspect, the system includes a third ultrasound treatment head having a third focal length and comprising a third plurality of transducer elements each having a third surface area different than the first surface area and the second surface area, the third plurality of transducer elements being configured to resonate at the desired operating frequency when the first ultrasound treatment head is electrically coupled to the generator.

In another aspect, the third plurality of transducer elements are configured to resonate at the desired operating frequency without requiring tuning with at least one capacitor.

An ultrasound system is also provided, comprising: a generator configured to generate histotripsy waveforms; a robotic positioning arm comprising a mechanical connection for physical attachment to a plurality of ultrasound treatment heads; an electrical connection for electrical coupling between the generator and the plurality of ultrasound treatment heads; a first ultrasound treatment head having a first focal length and comprising a first plurality of transducer elements each having a first surface area, the first plurality of transducer elements being configured to resonate at a desired operating frequency when the first ultrasound treatment head is electrically coupled to the generator; and a second ultrasound treatment head having a second focal length and comprising a second plurality of transducer elements each having a second surface area different than the first surface area of the first plurality of transducer elements, the second plurality of transducer elements being electrically coupled to at least one capacitor configured to tune the second plurality of transducer elements to resonate at the desired operating frequency when the second ultrasound treatment head is electrically coupled to the generator.

In additional aspects of the systems described above, the at least one capacitor is selected to tune the plurality of transducer elements to achieve the desired operating frequency that is lower or higher than what is specified for the second plurality of transducer elements based upon a thickness and material composition of the second plurality of transducer elements.

In some aspects of the systems described above, the at least one capacitor is selected to achieve a desired acoustic pulse shape.

In additional aspects of the systems described above, the at least one capacitor has a capacitance ranging from 1 to 1000 pF.

In one aspect of the systems described above, the at least one capacitor includes more than one capacitance to produce a multi-frequency transducer array.

In other aspects of the systems described above, the at least one capacitor is disposed on a printed circuit board (PCB) that is electrically coupled to the generator and one or more transducer elements.

In some aspects of the systems described above, the at least one capacitor forms a parallel connection between a positive connection and a ground connection to each transducer element.

In additional aspects of the systems described above, the plurality of transducer elements have the same surface area.

In other aspects of the systems described above, the plurality transducer elements have varying shapes.

A transducer array is provided, comprising: an array shell comprising concave distal surface; a plurality of transducer elements disposed in the array shell and configured to transmit ultrasound energy through the concave distal surface towards a common focal point; and a back cover coupled to a proximal surface of the array shell, the back cover and array shell comprising an interface in which a clearance or gap is formed between a majority of the array shell and back cover to reduce forces applied by the back cover against the array shell.

In one aspect, the array includes an adhesive disposed in the clearance or gap.

In another aspect, the array comprises a lap-joint connection between the array shell and the back cover.

In other aspects, the array comprises a snap or friction fit between the array shell and the back cover.

In some aspects, the clearance or gap is configured such that the back cover minimizes or does not induce any downward force on the array shell.

A method of manufacturing a transducer array is provided, comprising: machining an emitting surface of an array shell out of a solid piece of material; machining a plurality of wells into a rear surface of the array shell; inserting a transducer element into each of the plurality of wells of the rear surface; and providing an electrical connection to each transducer element.

In one aspect, the array shell is machined out of a plastic material.

In another aspect, the method comprises, prior to inserting the transducer elements, inserting a matching layer into each of the plurality of wells.

In some aspects, the method includes applying an adhesive layer between each matching layer and transducer element for a given well.

In another aspect, the method includes providing the electrical connection comprises attaching a wire to each transducer element.

In some aspects, the method includes providing the electrical connection comprises attaching a wire to a front surface of each transducer element via a notch formed in the transducer element.

In some aspects, the emitting surface is concave.

In another aspect, the method includes applying an encapsulating layer on top of each transducer element.

In one aspect, the method includes machining flat facets in the emitting surface corresponding to each of the plurality of wells.

In some aspects, the plurality of wells have varying shapes.

In another aspect, the plurality of wells have the same surface area.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIGS. 2A-2C show one example of a therapy transducer array for a histotripsy system.

FIGS. 3A-3C show cross-sectional views of wells of an array shell including the matching layer and transducer element components that reside within the wells.

FIGS. 4A-4B show additional views of transducer elements and matching layers.

FIGS. 5A-5G show a first embodiment of a histotripsy transducer array.

FIGS. 6A-6E show a second embodiment of a histotripsy transducer array.

FIGS. 7A-7E show a third embodiment of a histotripsy transducer array.

FIGS. 8A-8D show another embodiment of a transducer array.

FIGS. 9A-9C are schematic illustrations of an inductive current driver circuit for a histotripsy signal generator.

FIG. 10 is a plot showing a single array element acoustic pulse shape generated by an inductive current driver circuit with different combinations of inductor and capacitor values.

FIG. 11 is a system diagram showing a histotripsy therapy system that includes a therapy cart, a fluidics cart, and a coupling system.

FIG. 12 is another view of a coupling system of a histotripsy therapy system.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

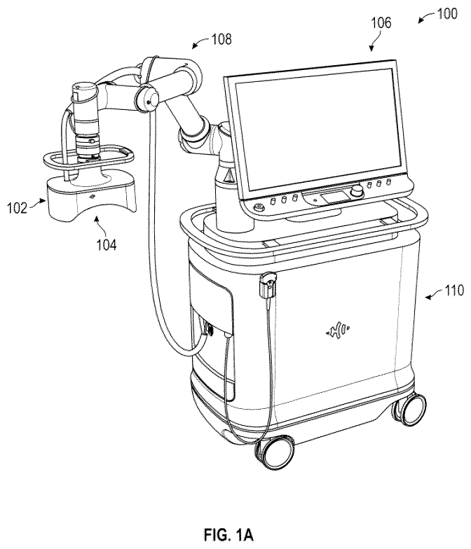

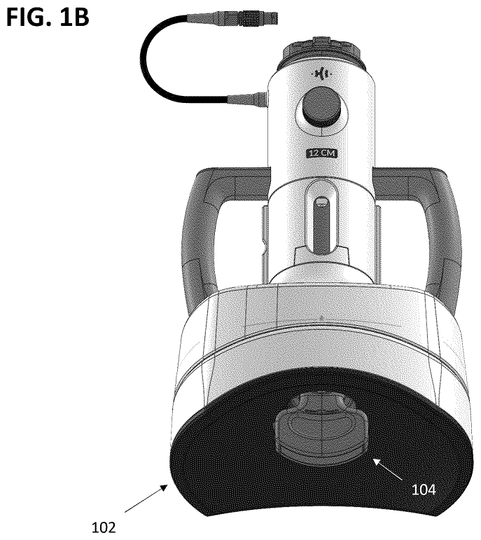

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image. In other non-illustrated embodiments, the therapy transducer does not include an opening 204 for the imaging system.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ˜24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows or expands (the bubble cloud) towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHZ, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

### Therapy Transducer

The therapy transducer can comprise a single element or multiple elements configured to be excited with high amplitude electric pulses. The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. As used herein, the term “acoustic stack” refers to the components within a transducer array that can include the transducer element, at least one matching layer, and may further include one or more adhesive layers to bond together the individual components. Further, the piezoelectric material can be of polycrystalline/ceramic or single crystalline formulation. In some embodiments the transducer elements can be formed using silicon using MEMs technology, including CMUT and PMUT designs.

### Transducer Shell

According to one aspect of the disclosure, a novel ultrasound transducer is provided which is purpose-built for Histotripsy therapy and includes features which enable easier and cost-effective manufacturing overcoming many of the limitations and challenges of traditional transducer manufacturing discussed above, while also being capable of and configured to produce ultrasound wavefronts that are optimized for histotripsy therapy and cavitation bubble formation.

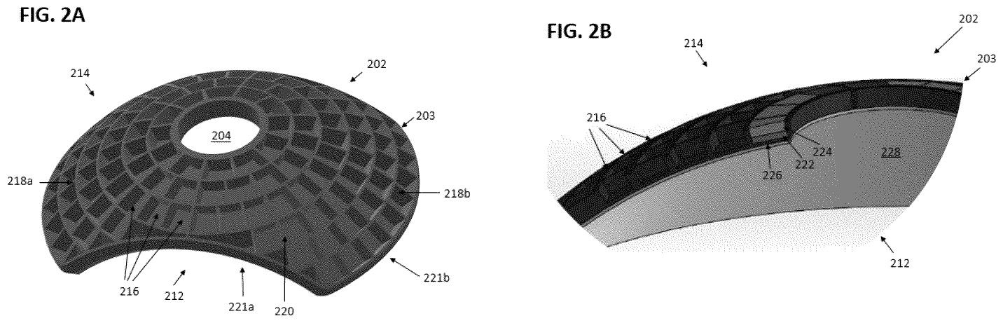

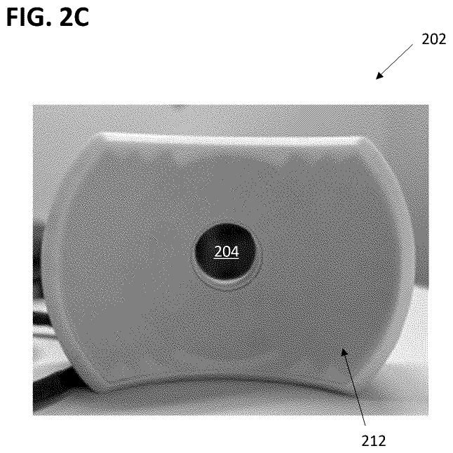

Referring to FIGS. 2A-2C, a histotripsy therapy transducer array 202 can include an array shell 203 that includes an opening or aperture 204 to accommodate an imaging probe or imaging system, as described above in FIG. 1B. The array shell can be spherically shaped and can include, for example, a first, concave (therapy) emitting surface 212 on the front or distal side of the array shell (relative to the robotic arm) and a second, rear surface 214 that includes a plurality of wells 216 or openings formed therein. FIG. 2C shows another view of a histotripsy therapy transducer array 202 including the first, concave emitting surface 212 and an opening or aperture 204 shaped to receive an imaging transducer. In certain embodiments, the array shell may also be cylindrical or ellipsoidal. The emitting surface can be formed of a solid, smooth, contiguous material. In other embodiments, the emitting surface may have flat facets corresponding to or matching each of the wells on the rear surface of the array shell.

The wells 216 in the rear surface of the array shell can comprise any shape, but in the illustrated embodiment the bottom or lower surface of individual wells are generally rectangular, trapezoidal, or square in cross-section and in shape and several wells together may comprise rings or ring segments. Generally, the thickness of the array shell in front of each well is uniform in thickness. However it should be understood that in some embodiments, different wells corresponding to different transducer elements may have different emitting surface thicknesses. It should be understood that the wells can also comprise circular, oval, or any other regular or irregular shapes/sizes and combinations thereof as needed for a particular transducer design. In some embodiments, the surface area of the bottom or lower surface of the wells is similar or the same, allowing for all transducer elements in the array to have the same or substantially equivalent emitting surface areas. Specifically, the surface area of all the wells within one ring or within one ring segment is similar/may be the same. In particular embodiments, the bottom cross-sectional area of the wells and therefore the transducer elements can be the same across the entire array, even if individual elements/wells have different cross-sectional shapes. This allows for generator electrical matching circuits and the subsequent pulses produced by the transducer elements to be the same or substantially equivalent.

Furthermore, the wells 216 can be arranged in one or more physical groupings within the array shell. In the illustrated example, the wells are arranged in two halves or groups 218a-218b. Each half comprising semi-circular rings, the two halves being mirror images of one another. The wells within each semi-circular ring are of the same or substantially the same bottom surface cross-sectional shape and area. The number and arrangement of groups depends on the overall shape of the array shell and the desired number of transducer elements. For example, the embodiment of FIG. 2A includes a plurality of “full” rings (e.g., three full rings) and a plurality of “partial rings” (e.g., three partial rings that don't fully extend in a complete circle) since the array shell includes two straight edges 221a and two rounded edges 221b. Additionally, the physical grouping of the transducer elements is not determinative on how the transducer elements themselves are driven or excited. For example, while the transducer elements can be driven or excited according to the physical groupings, they can also be driven individually by the driving electronics of the system, or according to other groupings unaffiliated with the physical grouping. For example, the transducer elements may be driven in groupings of concentric rings or other predetermined or user selected groupings depending on the target tissue being treated and other treatment planning or patient specific factors.

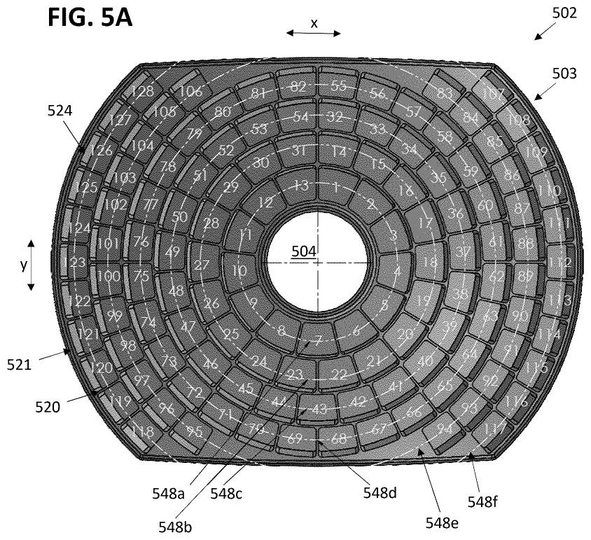

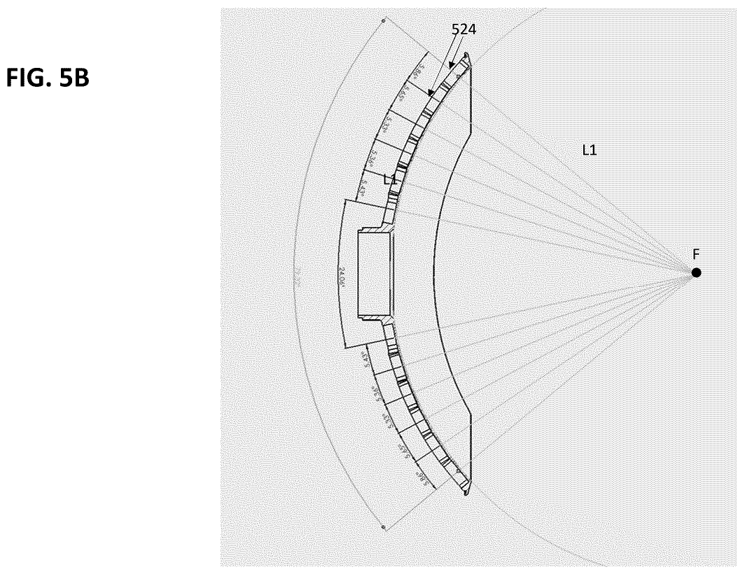

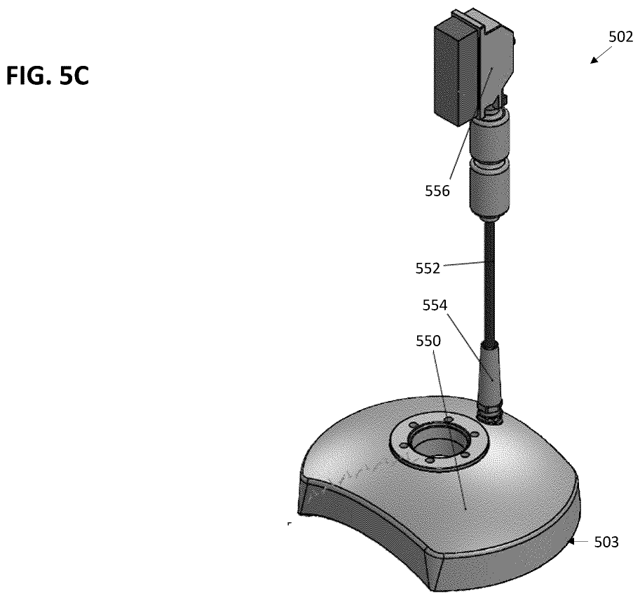

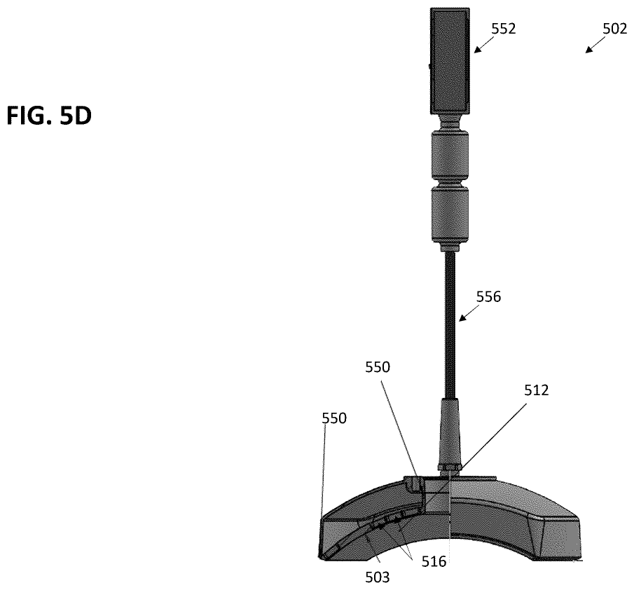

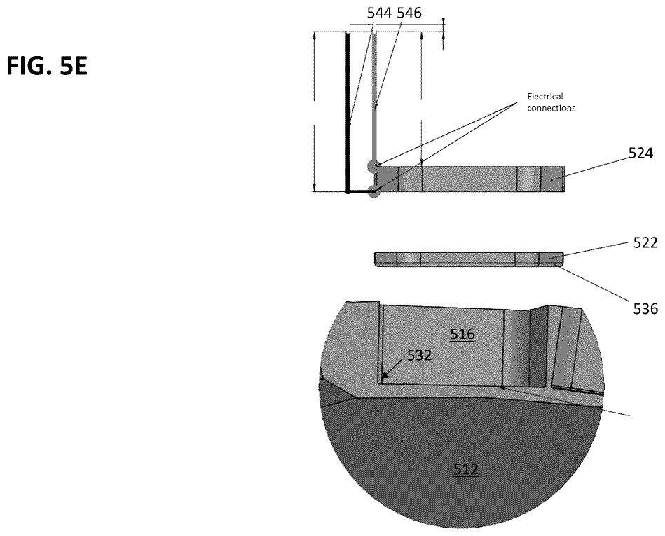

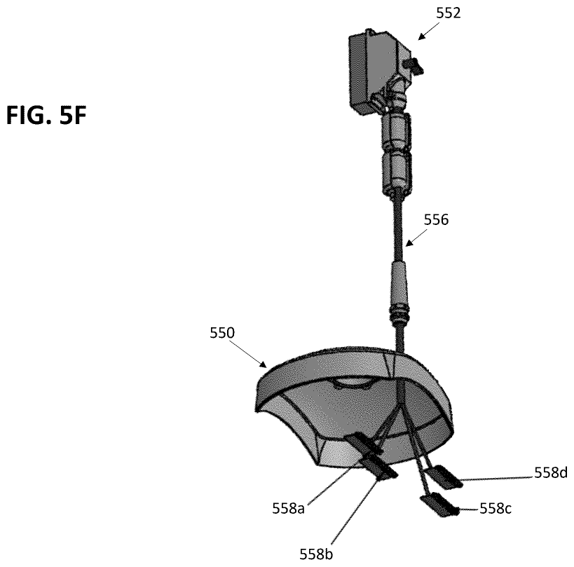

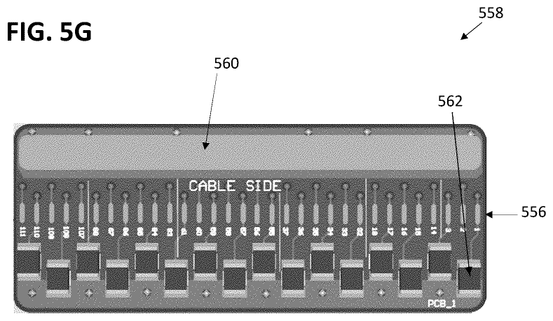

In particular, as illustrated in at least FIGS. 2A, 5A, 6A and 7A the wells are arranged in concentric rings or ring segments. In FIG. 5A, the first ring, for example, may comprise 13 individual wells, the bottom or lower surface of each well having similar or identical cross-sectional areas and shapes within the first ring, labelled as wells 1-13. The second ring may comprise 18 individual wells, wherein the bottom or lower surface of each well having similar cross-sectional areas and shapes within the second ring, labelled as wells 14-31. The cross-sectional area and shape of the bottom or lower surface of the first ring wells may be the same as or different than the cross-sectional area and shape of the bottom or lower surface of the second ring wells. Similarly, the cross-sectional area and shape of the bottom or lower surface of the wells of each individual ring or ring segment may be the same as or different than one another. The third ring may include wells 32-54 for a total of 23 individual wells, all having a bottom or lower surface of similar cross-sectional area and shape. The fourth ring includes wells 55-82 for a total 28 wells, all having a bottom or lower surface of similar cross-sectional area and shape. The fifth and sixth rings each form ring segments, with 24 wells and 22 wells respectively. The bottom or lower surface of wells in the fifth ring segment are all of similar cross-sectional area and shape and the wells of the sixth ring have bottom or lower surfaces of similar cross-sectional area and shape. Each of the transducers 502,602, and 702 in FIGS. 5A, 6A, and 7A, respectively, can house 128 transducer elements, each positionable within the 128 wells of the array shell. In particular, one acoustic stack may also seated within each well, for a total of 128 acoustic stacks.

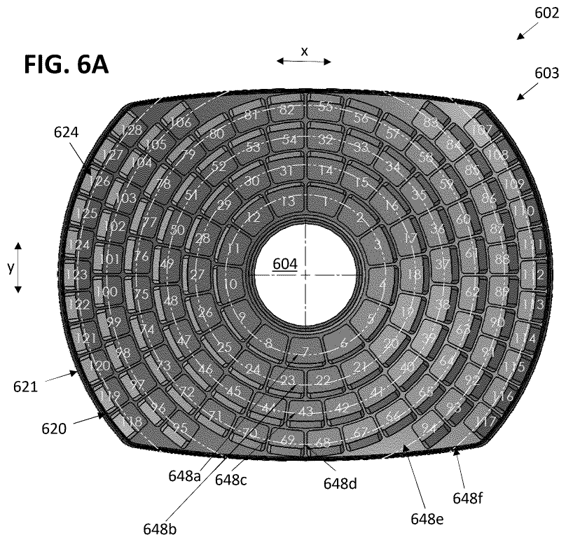

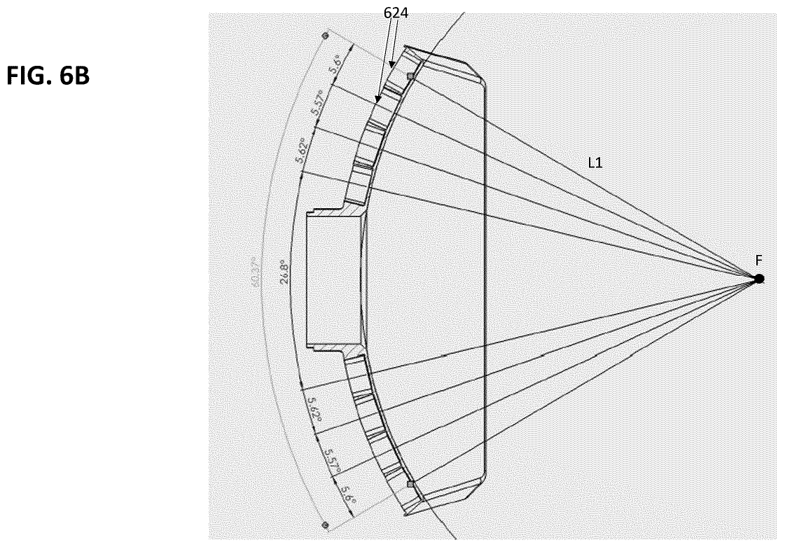

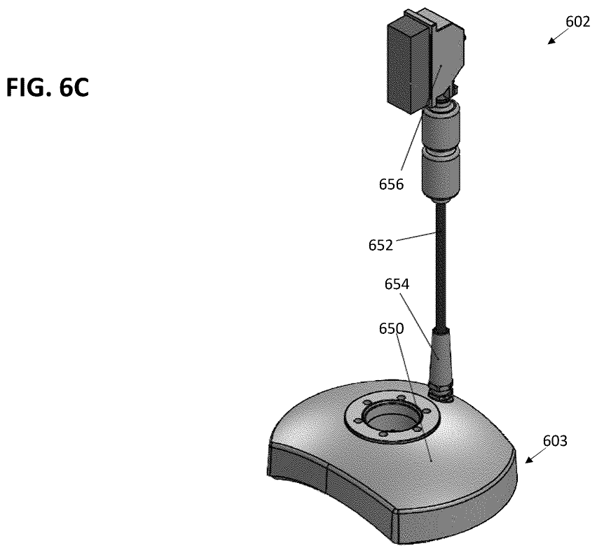

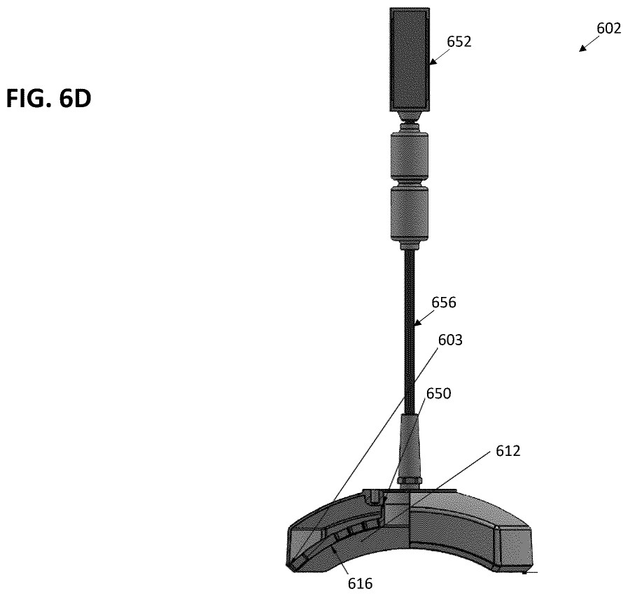

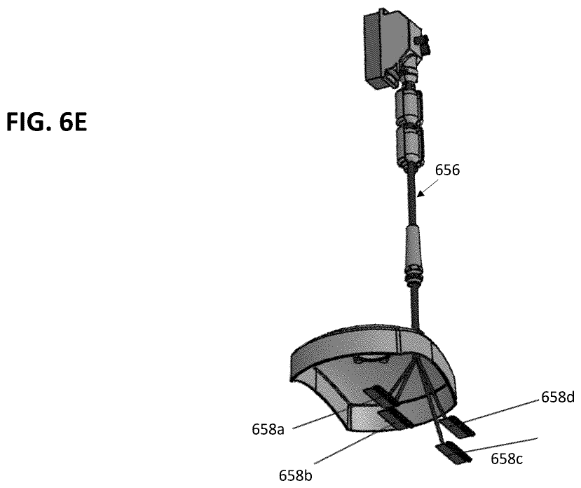

FIG. 6A has the same number of rings, ring segments and wells in each ring or ring segment as FIG. 5A. As such, the above description applies to FIG. 6A. It should be noted that the cross-sectional area and shape of the bottom or lower surface of wells for the transducer array illustrated in FIG. 6A may be the same as or different than the cross-sectional area and shape of the wells for other transducer arrays disclosed herein. As described herein, the wells in each ring or ring segment may have bottom or lower surfaces with the same or similar cross-sectional area. In embodiments, the cross-sectional area of the bottom or lower surface of the wells or the first ring may be different than or the same as the cross-sectional area of the bottom or lower surface of wells of the second, third, fourth, fifth, and/or sixth rings. It should also be noted that the left and right halves in the Y direction can be mirror images of one another. One transducer element will be seated within one well, for a total of 128 transducer elements. In particular, one acoustic stack may also seated within each well, for a total of 128 acoustic stacks.

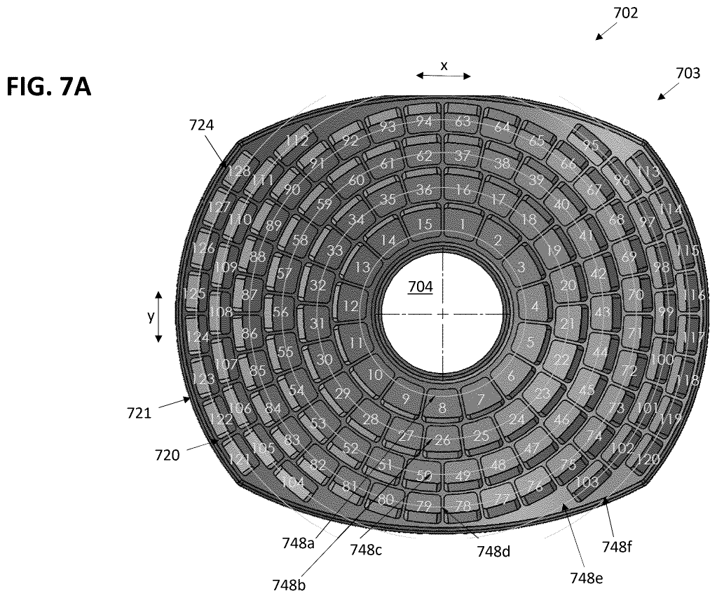

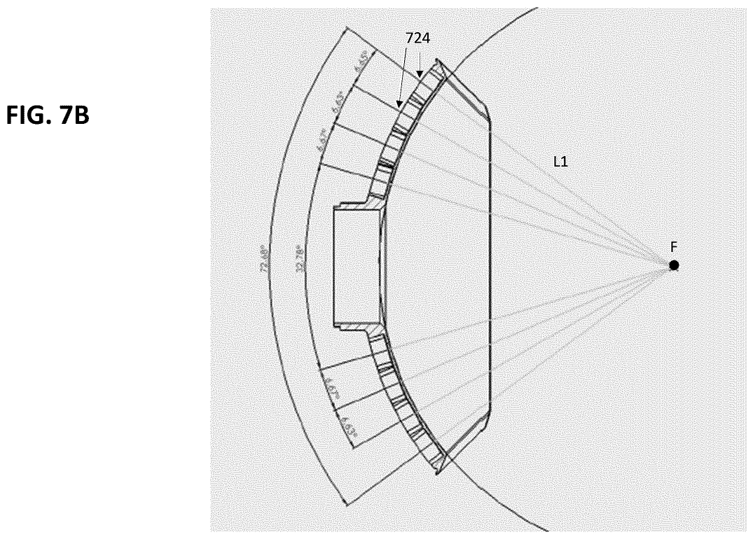

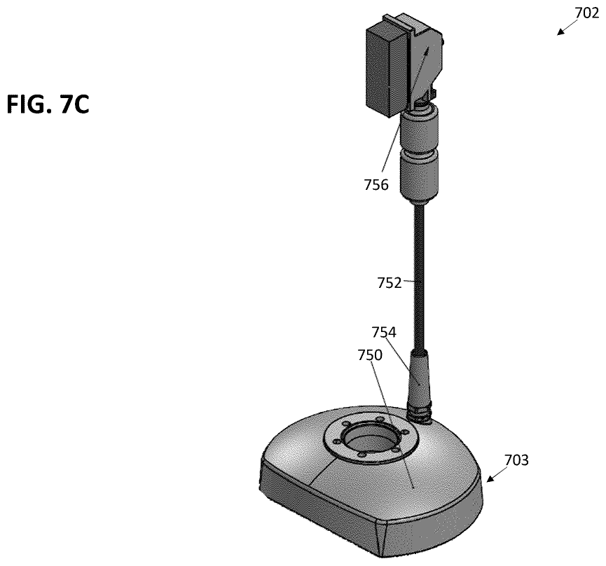

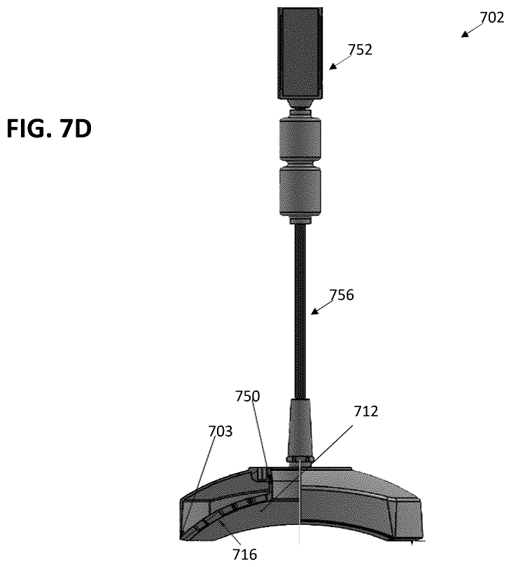

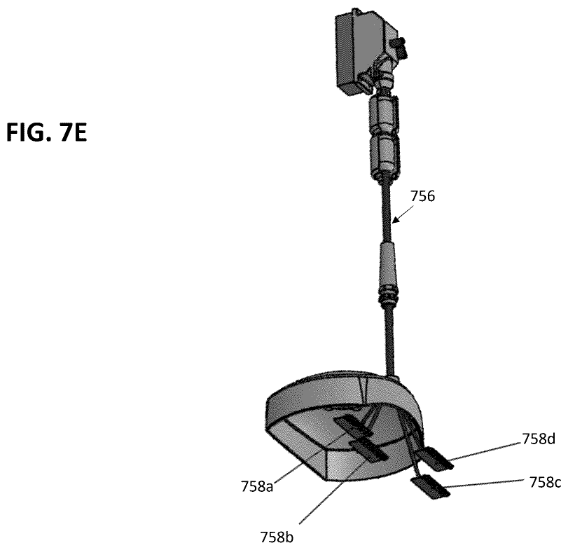

FIG. 7A includes a different arrangement in terms of number of wells within each concentric ring and ring segment of the array shell. This transducer array, having a smaller diameter than the transducer arrays above, can be intended for shallow targets. In particular, the first ring, for example, may comprise 15 individual wells, all having bottom or lower surfaces with similar cross-sectional areas and shapes within the first ring, labelled as wells 1-15. The first ring as described includes the ring closest to the central portion of the transducer. The second ring may comprise 17 individual wells, all having bottom or lower surfaces with similar cross-sectional areas and shapes within this second ring, labelled 16-36. The bottom or lower surface cross-sectional area and shape of the first ring wells may be the same as or different than the bottom or lower surface cross-sectional area and shape of the second ring wells. Similarly, the bottom or lower surface cross-sectional area and shape of the wells of each individual ring or ring segment may be the same as or different than one another. The third ring may include wells 37-62 for a total of 26 individual wells, all having bottom or lower surfaces with similar cross-sectional areas and shapes. The fourth ring includes wells 63-94 for a total 31 wells, all having bottom or lower surfaces with similar cross-sectional areas and shapes. The fifth and sixth rings each form ring segments, with 18 wells and 16 wells, respectively. The wells in the fifth ring segment are all have bottom or lower surfaces with similar cross-sectional areas and shapes, and the wells of the sixth ring segment have bottom or lower surfaces of similar cross-sectional area and shape. A total of 128 transducer elements will populate the 128 wells in this transducer array. In particular, one acoustic stack may also seated within each well, for a total of 128 acoustic stacks.

In one embodiment, the array shell can be machined from a single piece of polymer such as polyethylene terephthalate (PET) or plastics such as polyethylene ether keytone (PEEK), polypropylene (PP), poly acrylamide (PA), acrylonitrile butadiene styrene (ABS) are also envisioned. In some examples, all of the concave emitting surface 212, the rear surface 214, and the wells 216 can be machined out of the polymer or plastic material. Machining the array shell provides for precision and cost-effective manufacturing that allows for precisely controlling the thickness and shape of the emitting surface, the well to well curvature, the distribution of material between the wells and the emitting surface, and the ability to provide very thin separating walls 220 between each adjacent well. Additional methods for manufacturing the array shell may include molding, 3D printing, casting and combinations thereof, however machining the array has shown advantages compared to the other techniques and methods of manufacturing.

The array shell may further contain additives including but not limited to plasticizers, hardeners, fire-resistance/fire-proofing, acoustic enhancement, impact resistance, and the like. Additives may be applied in the form of a coating, co-extruded, mixed into the PET (or other polymer comprising or partially comprising array shell) or otherwise added into, therein or thereon the array shell or transducer.

### Transducer Elements and Matching Layers

The embodiment of FIG. 2A shows the array shell 203 without any transducer elements. FIG. 2B shows a cross-sectional view of the histotripsy therapy transducer array 202 including the matching layer(s) and transducer elements. For example, each well 216 of the array shell 203 can be filled with a matching layer 222 and individual transducer elements 224 on top of the matching layer 222. Additionally, the array shell itself acts as a matching layer, so the portion of the array shell between each well and the emitting surface 212 of the transducer array, labeled here as matching layer 226, is disposed between the transducer element 224 and the target tissue. Additionally and optionally, a third matching layer 228 may be applied onto the emitting surface of the array shell if additional matching is desired or required. The third matching layer may be, for example an encapsulating layer comprising an epoxy or urethane.

However, for purposes of this disclosure, the surface of the array shell is referred to as the emitting surface.

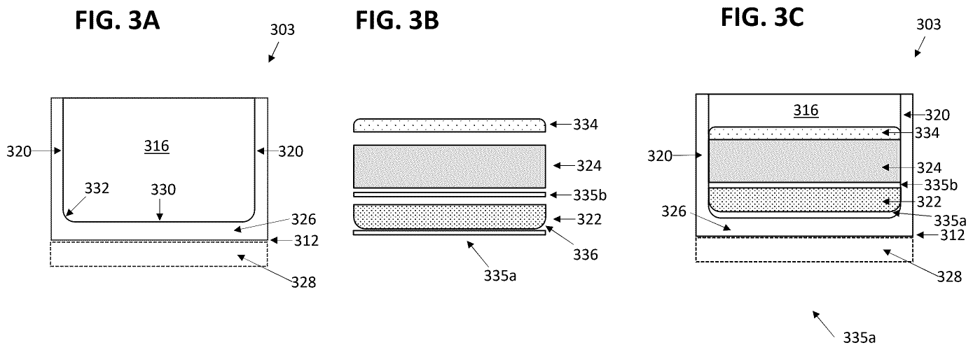

FIGS. 3A-3C give additional cross-sectional views of an array shell 303 including the wells 316 and the transducer element, matching layer, and encapsulating layer disposed therein. As previously described, the transducer element, at least one matching layer and the encapsulating layer may together comprise an acoustic stack. In FIG. 3A, a single well 316 is shown machined out of the array shell 303 including separating walls 320 between adjacent wells. As shown, the material of the array shell between the emitting surface 312 and the bottom surface 330 of the well provides a (second) matching layer 326 integrated directly into the array shell. In some embodiments, the wells include a bottom surface 330 that is flat, and optionally has rounded or scalloped corners or edges 332 as illustrated. However, it is envisioned that the interior edges may include interior angles of about 90 degrees or other varying angles. The array shell can further include an optional matching layer 328 disposed directly on the emitting surface 312. However, as described above, this matching layer is optional. The optional layer may provide additional acoustic impedance at a numerical value between that of the second matching layer and that of the acoustic coupling medium, which may be water or water-based.

Referring to FIG. 3B, the individual components that are placed within the wells of the array shell are shown. Specifically, a matching layer 322, a transducer element 324, and an optional adhesive 335a/335b or encapsulating layer 334 are illustrated. In some examples, the optional adhesive or encapsulating layer is not included in the wells of the array shell. It is noted that the matching layer 322 can include rounded edges 336 designed and configured to match the rounded corner edges 332 of the wells 316 of the array shell. Specifically, the radius of curvature of rounded edges 336 matches or is similar to the rounded corner edges 332 of the array shell wells 316. This ensures a precise fit and optimal matching and acoustic transmission through the matching layer and array shell during use. Additionally, as described above, the wells may include an adhesive or epoxy 335a and 335b disposed between the well of the array shell and the matching layer 322, and/or between the matching layer 322 and the transducer element 324, respectively. The adhesive or epoxy may also be disposed along other exterior portions of the transducer element and/or matching layer and/or well(s). The illustrated components in FIG. 3B, including the matching layer 322, transducer element 324, and array shell 326 can be collectively referred to herein as an “acoustic stack”. Optionally the acoustic stack can have an air backing or thin layer of adhesive between the transducer element 324 and air.

FIG. 3C shows an acoustic stack including the matching layer 322, transducer element 324, adhesive or encapsulating layer 334, and adhesive or epoxy layers 335a and 335b disposed within the well 316. The adhesive layers 335a/b may be disposed between each component placed in the well, in addition to the adhesive or encapsulating layer 334 on the periphery of the transducer element. The adhesive layers may comprise the same adhesive or different adhesives. For example, adhesive layers 335a and 335b are also shown in FIGS. 3B and 3C. The adhesive layers can be used to bond the various components together as well as eliminating air bubbles or other imperfections between layers that could potentially affect ultrasound transmission. As shown, when fully assembled, the matching layers 322 and 326 provide first and second matching layers (322 and 326, respectively) between the transducer element 324 and the emitting surface 312. An optional matching layer 328 disposed on the emitting surface can provide an additional matching layer if needed. In the illustrated embodiment, the matching layers and transducer elements are sized to fit precisely within the wells and fill out the entire interior diameter/surface area of the wells (e.g., the outer dimensions of the transducer elements precisely or exactly fit the interior dimensions of each well).

Matching layers 322, 422, etc. can comprise a polymer composite having glass or

ceramic particles/powder and have an acoustic impedance of about 5-8 Megarayl (Mrayl). In particular, the matching layer may be a glass or ceramic filled polymer such as perFORM™ manufactured by SOMOS® and can have an acoustic impedance of about 5.6 Mrayls. This matching layer may have a thickness of from about 0.5 mm to 1.5 mm, or optionally from about 1.3 mm+−0.13 mm. Other matching layers may also be used, including but not limited to glass or soda-lime glass. In some examples, more than one matching layer, or matching layers of different materials (e.g., perFORM, glass, etc.) may be used in place of the described matching layers. Transducer elements 324,424 may have an acoustic impedance of between 10-36 MRayls, and in some embodiments approximately 20 MRayls, and can include a conductive coating such as Gold, Silver or Nickel. In one embodiment, the transducer element may comprise a Gold coating on at least one side. The transducer element may include a thickness of about 2.35 mm+−. 01 mm. The adhesive layer may be a low viscosity epoxy.

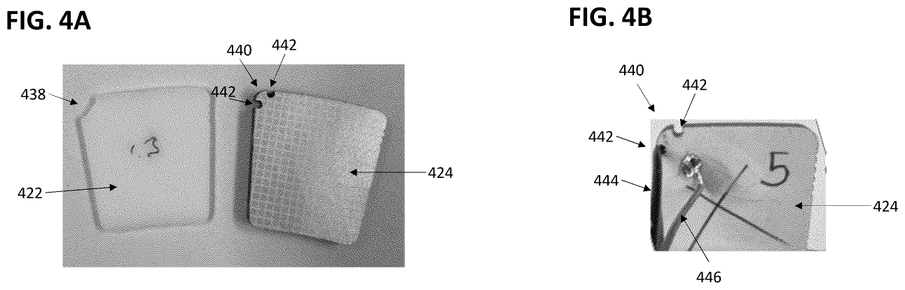

FIGS. 4A-4B show additional views of a matching layer 422 and transducer element 424, which can correspond with matching layer 322 and transducer element 324 of FIGS. 3A-3C. As shown in FIG. 4A, the matching layer 422 can include a notch or cutout 438 from one or more of the corners of the matching layer. This notch or cutout is designed and configured to align with connection interface 440 of the transducer element 424 when the matching layer and transducer element are placed in a well of an array shell. The connection interface 440 of the transducer element can further include one or more cutouts or notches 442 which can permit passage of one or more wires to the front face of the transducer element. The notch or cutout 442 is shaped to receive one or more wires. Matching layers and transducer elements may further include additional markings for ease of assembly or manufacturing.

The notch or cutout 438 in the matching layer 422 is designed and configured to accommodate a solder connection to the front face of the connection interface 440 (e.g., the face of the transducer element that rests against the matching layer). FIG. 4B shows a transducer element 424 with first electrical connection 444 (e.g., negative or ground voltage connection) passing through cutout or notch 442 in the transducer element to the front face of the piezo element, and second electrical connection 446 (e.g., positive voltage connection) is soldered directly to the back face of the transducer element. The notch or cutout 438 in the matching layer allows for routing the first electrical connection 444 to the front face of the piezo element while allowing the remainder of the transducer element to rest flush or flat against the matching layer 422 and or the adhesive layer at the bottom of the well (e.g., adhesive layer 335a). The first and second electrical connections are attached at a second end to at least one PCB board, which will be described in more detail below.

In an alternative embodiment, more than one electrical connection can be made to the transducer element to combat resistance across the element 424. The transducer element 424 can be separated into two or more separate transducer elements with one or more ground connections and a positive electrical connection to each. This provides the ability to increase array element count without increasing the number of array shell wells (e.g. two electrically separate positive electrodes on the transducer element material in one well would double the number of array elements).

Once the electrical connections are made to the transducer element, and the matching layer 422 and transducer element 424 are placed within a well of an array shell, an additional adhesive or encapsulating layer (e.g., encapsulating layer 334 in FIG. 3B) can be placed within or over the component stack to electrically seal everything and hold the components in place within the well. Referring to FIG. 4B, the other cutout or notch 442 that is not used by the first electrical connection 444 can be used as a guide for applying the adhesive or encapsulating layer between solid layers of the acoustic stack.

### Therapy Treatment Head Designs

The principles described above with respect to transducer array design and manufacturing can be implemented in a variety of therapy treatment head sizes all configured to work seamlessly with a single signal generator and amplifier (e.g., driving electronics). Described herein are three distinct treatment head designs with sizes ranging from 14 cm for the smallest array up to 20 cm for the largest array. Moreover, the principles described herein could be applied towards smaller or larger arrays, such as, for example, arrays ranging between 8-11 cm or larger than 20 cm. The treatment heads can be hot-swappable onto a robotic arm and selected by a physician based on the target tissue, tissue type, intervening anatomical structures, treatment depth, and/or size of the volume to be treated.

FIG. 5A shows a first embodiment of a histotripsy transducer array 502 in a 20 centimeter (cm) configuration. The 20 cm configuration indicates the radius of curvature of the face of the transducer array, as well as the natural focal length L1 of the transducer array. FIG. 5B illustrates in more detail, the natural focal length L1, which may be defined as the distance or length from the transducer element or transducer array to the natural focus (F) of a target location. In particular, acoustic stacks are arranged with the array shell such that the focal length L1 of each transducer element is 20 cm. Further, centers of the individual transducer elements 524 are offset from one another by from about 4 degrees to about 7 degrees, with specific offsets for the illustrated embodiment shown in the figure. As shown in FIG. 5A, the transducer array can include a total of 128 transducer elements 524 arranged in 6 rings or partial rings/ring segments around the opening or aperture 504 in the array shell 503. The transducer elements (numbered 1-128 in at least FIGS. 5A, 6A and 7A) can be disposed within wells of the array shell as previously described, the wells and transducer elements oriented to direct ultrasound energy from the transducer elements towards a common focal point F. As shown in FIG. 5A, the transducer elements can be arranged in full rings 548a-548d, and partial rings 548e-548f, resulting in a transducer array with a longer dimension along the x axis compared to the y axis.

As shown in FIG. 5A, the precision milling of the array shell 503 and the arrangement of transducer elements 524 into wells facilitates very efficient use of the surface area of the transducer array, allowing for the transducer elements to make up a very high (e.g., 85% or higher) percentage of the overall surface area of the array shell. The milling of the wells in the array shell provides for very thin (e.g., 0.5 mm or less) walls 520 between adjacent transducer elements, and also provides for a very thin (e.g., 2 mm or less) bezel 521 around the external perimeter of the array shell allows for deeper treatment in tissue compared to prior histotripsy transducer arrays with much thicker bezels

FIG. 5C shows additional details of the transducer array 502, including array shell 503, back cover 550, cable assembly 552, strain relief 554, and cable connector 556. The cable assembly 552 provides electrical connections between each of the transducer elements of the array 502 to a signal generator and amplifier of the histotripsy system (not shown). The cable connector provides the physical connection between the cable assembly and an interface on the histotripsy system.

FIG. 5D shows the back cover 550 adhered to the array shell 503, including a cross-sectional view of one or more wells 516. With the transducer array 502 only comprising two main components, the array shell and the back cover, alignment between the focus of the therapy transducer array and an imaging probe (inserted through the aperture or opening of the array) can be improved.

In FIG. 5E another cross-sectional view of a well 516 is shown opposite the emitting surface 512. As described above, the well can be machined resulting in rounded or scalloped edges 532, particularly at the bottom of the well. The transducer array components (acoustic stack) that reside within the well are also shown, including the matching layer 522 that includes rounded or scalloped edges 536 designed and configured to precisely match the rounded or scalloped edges of the well, and a transducer element 524 configured to be disposed on top of the matching layer 522. First and second electrical connections 544 and 546 are also shown, with the corresponding welds for attaching to the front and rear faces of the transducer element.

In FIG. 5F, a bottom-perspective view is provided which shows the cable assembly 556 split into groupings of cable assemblies which each terminate at separate interconnect arrays (e.g., printed circuit boards (PCBs)) 558a-558d. As shown, the PCBs can be disposed under the back cover 550, between the back cover and the array shell. It should be understood that the PCBs could be located in other locations between the electrical path from the generator to the transducer element. In some examples, the PCBs are located in or near the generator, away from the transducer element. In other embodiments, the PCBs may located, for example, within the cable assembly 552. In some embodiments, the number of PCBs can coincide with the number of physical groupings of transducer elements. While the illustrated embodiment includes 4 groupings of transducer elements resulting in 4 PCBs, it should be understood that any number of groupings and PCBs can be implemented. The PCBs provide individual electrical connections between the channels of the signal generator/amplifier and each transducer element. Therefore, a 128 element transducer array divided into 4 separate groupings results in each PCB managing the electrical connections from a 128 channel signal generator/amplifier to 32 separate transducer elements. It should be understood that a single PCB can be used for each transducer element (e.g., 128 PCBs for 128 transducer elements), or alternatively a single PCB could be used for all transducer elements. The number of transducer groupings can also be varied, such as 4 groupings, 8 groupings, 16 groupings, etc. for a 128 element transducer array.

FIG. 5G is an example of a PCB 558 for electrically connecting the cable assembly and signal generator/amplifier of the histotripsy system to groups of transducer elements in the transducer array. As shown, the PCB can include electrical contact 560 for the ground connection to the cable assembly, and individual signal connections to each of the elements in the grouping. The PCB further includes a plurality of tuning pads 562 configured to be electrically coupled to capacitors or inductors for tuning different transducer arrays to be optimized for use with a single signal generator. As will be described in further detail below, the ability to tune transducer arrays at the electrical connection interface (e.g., with the PCBs) allows for different sized transducer arrays and/or different sized and thickness transducer elements to be individually tuned without having to swap out the signal generator or provide separate signal generators optimized for each different transducer array.

In some embodiments, tuning the PCBs of FIGS. 5F and 5G can supplement the signal generator/amplifier matching circuit to tune the impedance for one or more transducer elements or for a set of transducer elements for a given transducer array. For example, additional parallel capacitance can be added to smaller transducer elements to make them resonate at the same, or substantially equivalent, frequency as larger transducer elements. Capacitance can also be varied to achieve a desired acoustic pulse shape and/or frequency lower or higher than what is specified based upon the thickness and composition of transducer elements and matching layer(s). In one example, the capacitance of the tuning capacitors can range from 1-1000 pF. Alternatively, different transducer elements within a given array can be tuned differently, including being tuned to different frequencies to achieve multi-frequency transmissions. Tuning the PCBs with capacitors therefore allows for different sized transducer arrays (e.g., 20 cm focal length, 17 cm focal length, 14 cm focal length, etc.) which may use different sized transducer elements to be tuned in a way that allows them to be used with a single driving circuit or architecture to achieve consistent and predictable results in tissue. In some embodiments, the tuning is used to optimize histotripsy pulse shape in tissue for a given transducer array/transducer element. Additionally, adding inductors to the PCB connections can be used to reduce the current needed to drive each element, and also provide benefits to the signals produced by reducing or suppressing high frequency noise/peaks. Additionally, adding inductors to the PCB connections can also be used to increase drive efficiency.

It should be understood that not all array transducer designs must include a capacitor or inductor mounted on the PCB for tuning. In one embodiment, a system having three different array sizes (e.g., 20/17/14 cm) may use capacitors to tune only two of the arrays, with the third array being optimized without the need for additional capacitive or inductive tuning. For example, in such a system, the 20 cm array may not have additional capacitors or inductors for tuning, but the 17 cm array, and the 14 cm array may include capacitors on their respective PCBs. Additionally, the capacitors used between the different sized arrays may be different (e.g., the 17 cm array may have a has a 360 pF capacitor while the 14 cm array may have a 680 pF capacitor). Generally speaking, a larger capacitor is needed to accommodate a higher transducer element electrical impedance. Note electrical impedance is inversely proportional to transducer element surface area assuming the same piezoelectric material is used in all designs.

Additionally, different piezoelectric materials can be used in this overall therapy subsystem design. In some embodiments, the material used for transducer elements in one of the arrays (e.g., the 20 cm transducer) may use a different piezoelectric composite material than the other transducer arrays (e.g., the 14 cm and 17 cm transducers). Other aspects of the acoustic stack can also be adjusted or modified for each transducer array to optimize each array (e.g., different matching layer thicknesses or materials.

The size of the array may also dictate the use of different piezoelectric composites. For example, composite materials with higher dielectric permittivity could be more suited for smaller and/or lower frequency elements and/or arrays. These smaller arrays may be suited for shallower tissue targets, such as thyroid or breast applications. Optimizing and tuning these transducer arrays with the appropriate piezoelectric for a given array/element size allows the arrays to have the same or similar frequency, and therefore be driven with the same generator matching circuit as the other transducer arrays of the system.

FIG. 6A shows a second embodiment of a histotripsy transducer array 602 in a 17 cm configuration. The 17 cm configuration indicates the radius of curvature of the face of the transducer array, as well as the natural focal length (L1) of the transducer array. FIG. 6B illustrates in more detail, the natural focal length L1, which may be defined as the distance or length from the transducer element or transducer array to the natural focus (F) of a target location. In particular, acoustic stacks are arranged with the array shell such that the focal length of each transducer element is 17 cm. Further, centers of the individual transducer elements 624 are offset from one another by from about 5 degrees to about 7 degrees, with specific offsets for the illustrated embodiment shown in the figure. As shown, the transducer array can include a total of 128 transducer elements 624 arranged in 6 rings or partial rings around the opening or aperture 604 in the array shell 603. The transducer elements can be disposed within wells of the array shell as previously described. As shown in FIG. 6A, the transducer elements can be arranged in full rings 648a-648d, and partial rings 648e-648f, resulting in a transducer array with a longer dimension along the x axis compared to the y axis.

As shown in FIG. 6A, the precision milling of the array shell 603 and the arrangement of transducer elements 624 into wells facilitates very efficient use of the surface area of the transducer array, allowing for the transducer elements to make up a very high (e.g., 87% or higher) percentage of the overall surface area of the array shell. The milling of the wells in the array shell provides for very thin (e.g., 0.5 mm) walls 620 between adjacent transducer elements, and also provides for a very (e.g., 2 mm) thin bezel 621 around the external perimeter of the array shell allows for deeper treatment in tissue compared to prior histotripsy transducer arrays with much thicker bezels.

FIG. 6C shows additional details of the transducer array 602, including array shell 603, back cover 650, cable assembly 652, strain relief 654, and cable connector 656. The cable assembly 652 provides electrical connections between each of the transducer elements of the array 602 to a signal generator and amplifier of the histotripsy system (not shown). The cable connector provides the physical connection between the cable assembly and an interface on the histotripsy system.

FIG. 6D shows the back cover 650 adhered to the array shell 603, including a cross-sectional view of one or more wells 616. With the transducer array 602 only comprising two main components, the array shell and the back cover, alignment between the focus of the therapy transducer array and an imaging probe (inserted through the aperture or opening of the array) can be improved.

In FIG. 6E, a bottom-perspective view is provided which shows the cable assembly 656 split into groupings of cable assemblies which each terminate at separate PCBs 658a-658d. In some embodiments, the number of PCBs can coincide with the number of physical groupings of transducer elements. While the illustrated embodiment includes 4 groupings of transducer elements resulting in 4 PCBs, it should be understood that any number of groupings and PCBs can be implemented. The PCBs provide individual electrical connections between the signal generator/amplifier and each transducer element. Therefore, a 128 element transducer array divided into 4 separate groupings results in each PCB managing the electrical connections to 32 separate transducer elements.

FIG. 7A shows a third embodiment of a histotripsy transducer array 702 in a 14 cm configuration. The 14 cm configuration indicates the radius of curvature of the face of the transducer array, as well as the natural focal length L1 of the transducer array. FIG. 7B illustrates in more detail, the natural focal length L1, which may be defined as the distance or length from the transducer element or transducer array to the natural focus (F) of a target location. In particular, acoustic stacks are arranged with the array shell such that the focal length L1 of each transducer element is 14 cm. Further, centers of the individual transducer elements 524 are offset from one another by from about 6 degrees to about 7 degrees, with specific offsets for the illustrated embodiment shown in the figure. As shown, the transducer array can include a total of 128 transducer elements 724 arranged in 6 rings or partial rings around the opening or aperture 704 in the array shell 703. The transducer elements can be disposed within wells of the array shell as previously described. As shown in FIG. 7A, the transducer elements can be arranged in full rings 748a-748d, and partial rings 748e-748f, resulting in a transducer array with a longer dimension along the x axis compared to the y axis.

As shown in FIG. 7A, the precision milling of the array shell 703 and the arrangement of transducer elements 724 into wells facilitates very efficient use of the surface area of the transducer array, allowing for the transducer elements to make up a very high (e.g., 85% or higher) percentage of the overall surface area of the array shell. The milling of the wells in the array shell provides for very thin (e.g., 0.5 mm) walls 720 between adjacent transducer elements, and also provides for a very (e.g., 2 mm) thin bezel 721 around the external perimeter of the array shell allows for deeper treatment in tissue compared to prior histotripsy transducer arrays with much thicker bezels.

FIG. 7C shows additional details of the transducer array 702, including array shell 703, back cover 750, cable assembly 752, strain relief 754, and cable connector 756. The cable assembly 652 provides electrical connections between each of the transducer elements of the array 602 to a signal generator and amplifier of the histotripsy system (not shown). The cable connector provides the physical connection between the cable assembly and an interface on the histotripsy system.

FIG. 7D shows the back cover 750 adhered to the array shell 703, including a cross-sectional view of one or more wells 716. With the transducer array 702 only comprising two main components, the array shell and the back cover, alignment between the therapy transducer array and an imaging probe (inserted through the aperture or opening of the array) can be improved.

In FIG. 7E, a bottom-perspective view is provided which shows the cable assembly 656 split into groupings of cable assemblies which each terminate at separate PCBs 758a-758d. In some embodiments, the number of PCBs can coincide with the number of physical groupings of transducer elements. While the illustrated embodiment includes 4 groupings of transducer elements resulting in 4 PCBs, it should be understood that any number of groupings and PCBs can be implemented. The PCBs provide individual electrical connections between the signal generator/amplifier and each transducer element. Therefore, a 128 element transducer array divided into 4 separate groupings results in each PCB managing the electrical connections to 32 separate transducer elements.

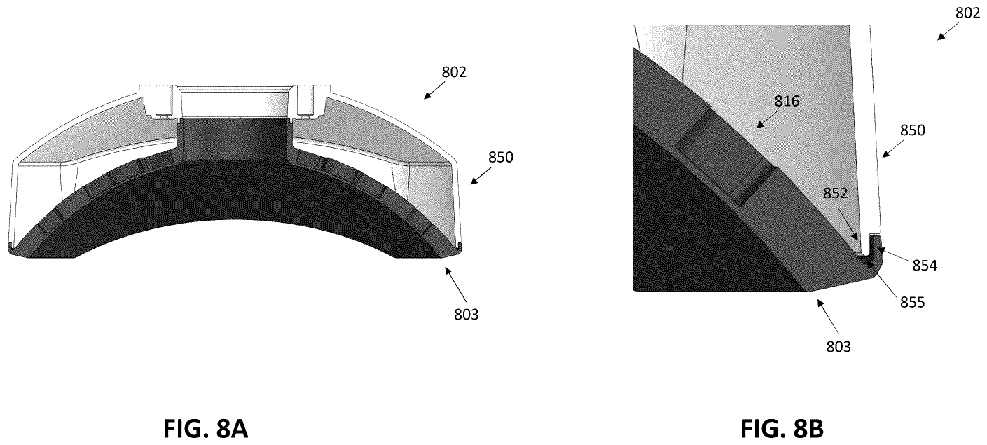

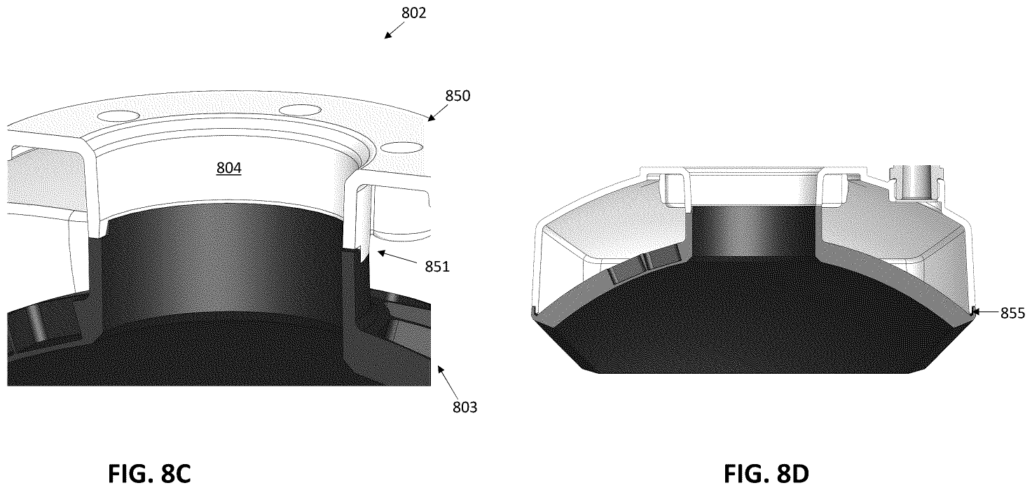

FIGS. 8A-8B show an cross-sectional view of a transducer array 802 including array shell 803 having a plurality of wells 816, and back cover 850. Cable assembly, strain relief, and cable connector are not illustrated. In this embodiment, the array shell 803 and the back cover 850 include a nested arrangement along a distal or front interface between the two components (e.g., the interface on the distal or patient facing end of the transducer array). As shown in FIG. 8B, in one example, the interface can comprise a tongue and groove, or lap-joint interface, in which a tongue or extension 852 in the back cover 850 is configured to engage with or be disposed within a corresponding groove or slot 854 in the array shell 803. This tongue and groove or lap-joint interface can be provided, for example, around a periphery of the transducer array.

In some embodiments, the interface between the back cover and the array shell can include an air gap or clearance 855 between the two components, as shown. The air gap or clearance is designed and configured such that the back cover minimizes or does not induce any downward force on the array shell, which would otherwise potentially change the curvature of the array shell (and therefore the overall geometry of the transducer array). The air gap may be filled with an adhesive to bond or seal the array shell to the back cover. Alternatively, the tongue or extension can be implemented in the array shell and the groove or slot can be integrated in the back cover, or any combination of tongue and grooves on either or both components can be used. Other friction fit, snap fit, or mechanical interfaces between two components as known in the art can also be implemented. An adhesive or epoxy may be utilized between the back cover 850 and array shell 803 to provide an additional seal between the two components.

FIGS. 8C-8D show additional views of a proximal interface of the transducer array 802, including keyed feature(s) 851 between the back cover 850 and the array shell 803 surrounding opening or aperture 804 in the back cover and array shell. In particular, the keyed feature includes a stepped arrangement between the back cover 850 and the array shell 803 adjacent to the opening or aperture 804 that accommodates the imaging probe assembly. FIG. 8C also shows the air gap or clearance between the back cover and the array shell as discussed above. Additional features may be included such as visual indicators for alignment between the various components of the back cover and/or array shell.

### Cart

The Cart 110 of the histotripsy system may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### FPGA and Other Therapy Components

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the transducer element. For example, because a transducer element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Inductive Driver Circuit

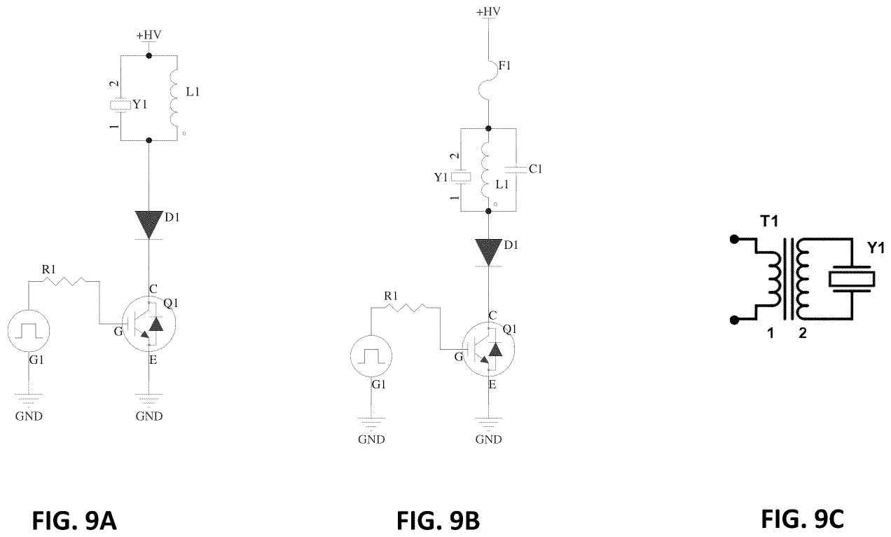

FIG. 9A depicts an inductive (current) driver circuit for driving a histotripsy therapy transducer array having a therapy transducer element Y1. The driver circuit shown in FIG. 9A describes a driver for a single channel/element. Therefore, a transducer array having 128 elements would have one of these drivers for each element (e.g., 128 inductive drivers). The driver circuit can include a charging inductor L1. The maximum driving voltage with this circuit is limited by the maximum operating voltage of the IGBT transistor Q1 and its switching time. Currently available IGBT transistors with the best performance for the inductive driving circuit is rated for a maximum of 3000V.

The inductive driver circuit uses the transducer element Y1 as a capacitor in parallel with the L1 inductor in order to create an oscillating circuit. When the IGBT transistor is excited with a single pulse, current flows through the inductor L1 which temporarily stores the energy in a magnetic field. As soon as the driving pulse disappears, this magnetic field is transferred back as a high amplitude electric pulse and this process keeps repeating at the resonant frequency of the LC circuit (L1 and Y1 in this case). The time that is needed for the inductor to be charged (aka. charging time) can vary and will proportionally affect the output amplitude (longer charge time equals higher output amplitude up to a 3,000 Volt maximum peak positive as described above). FIG. 9B shows another embodiment of an inductive current driver circuit in which a

capacitor C1 is added in parallel with the inductor L1 and the ultrasonic transducer Y1. The value of the capacitor C1 can be calculated to be as minimal as needed to have the oscillating circuit comprised of the inductor L1 and the capacitor C1 oscillating in the safe frequency and voltage ranges to prevent generation of voltages in excess to the working voltage of the transistor Q1 in case that the ultrasonic transducer gets disconnected or fails with open circuit.

The combined capacitance of the capacitor C1 and the transducer Y1 can be used to calculate the working frequency of the resonant circuit comprising the capacitor C1, inductor L1, and the ultrasonic transducer Y1. Alternatively, changing the value of the capacitor C1 can be used to fine-tune of the operating frequency of the entire circuit.

Referring to FIG. 9C, a step-up transformer may also be substituted for the inductor L1 in either inductive driver circuit. In this case the secondary winding (2) inductance would be the same value as L1.

As is noted above, the operating frequency of a histotripsy system can be fine-tuned and optimized not just by modifying or changing the capacitor values in the signal generator/driving electronics, but also by adjusting or applying capacitors at PCB connectors to the individual transducer elements. Therefore, a single inductive driver circuit can be used with fixed capacitance/capacitors C1, and individual transducer arrays can be fine tuned on the transducer-side PCBs with additional capacitance to adjust the operating frequency and/or other driving characteristics of the array. Tuning the capacitance of the transducer array at the electrical connections to the array itself, instead of at the electronic or inductive driver, allows for a single driving electronics to be used with multiple different transducer arrays, where each array can be fine-tuned based on the additional capacitance applied at the electrical connection/PCB.

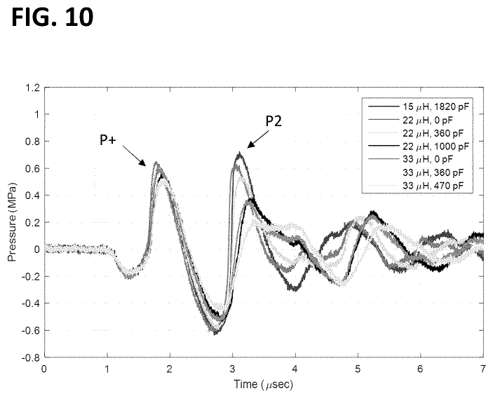

As noted above, the choice of inductor (L1) value, or inductor (L1) plus capacitor (C1) value, can affect the shape of the pressure pulse emitted by the therapy transducer element (Y1) of the histotripsy transducer array. FIG. 10 and Table 1 below shows measurements of pressure pulses emitted by a single therapy transducer element at a depth of 16 cm in water, illustrating the peak positive pressure (P+), peak negative pressure (P−) and trailing positive pressure (P2). In this example, the peak-to-peak voltage was kept constant (850 Vpp) for each choice of passive component matching. The general trend observed is that the trailing positive pressure (P2) and P2/P-Ratio decreased with an increase in LC matching circuit impedance. However, peak negative (P−) and positive (P+) pressure and pulse frequency decreased, and the VDC (direct current voltage) needed to achieve the same Vpp increased. This result demonstrates a significant trade-off in device headroom—the Vpp or VDC required to achieve a specific focal pressure—and trailing positive pressure half-cycle reduction

TABLE 1

| Inductor | Capacitor | VDC | P− | P+ | P2 | P2/P− | Frequency |
|---|---|---|---|---|---|---|---|
| (μH) | (pF) | (volts) | (MPa) | (MPa) | (MPa) | Ratio | (kHz) |
| 15 | 1820 | 48.5 | 0.64 | 0.72 | 0.72 | 1.14 | 745 |
| 22 | 0 | 48.0 | 0.61 | 0.65 | 0.63 | 1.03 | 761 |
| 22 | 360 | 49.0 | 0.60 | 0.61 | 0.54 | 0.91 | 747 |
| 22 | 1000 | 50.5 | 0.53 | 0.56 | 0.38 | 0.71 | 703 |
| 33 | 0 | 54.5 | 0.53 | 0.61 | 0.36 | 0.67 | 720 |
| 33 | 360 | 54.0 | 0.46 | 0.52 | 0.24 | 0.52 | 691 |
| 33 | 470 | 54.0 | 0.46 | 0.51 | 0.21 | 0.47 | 677 |

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a sealable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

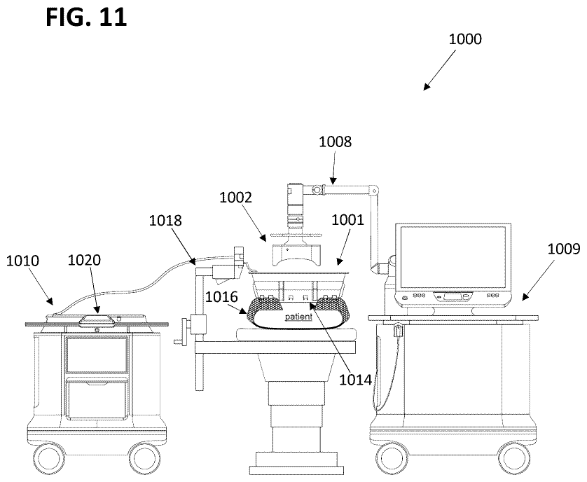

The disclosed histotripsy acoustic and patient coupling systems, in general, may comprise one or more of the following sub-systems and components, an example of which is depicted in at least FIG. 11, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

FIG. 11 illustrates one embodiment of a histotripsy therapy and imaging system 1000, including a coupling assembly 1001. As described above, a histotripsy therapy and imaging system can include a therapy transducer 1002, an imaging system 1004, a robotic positioning arm 1008, and a fluidics cart 1010. The robotic positioning arm may be attached to a therapy cart, such as cart 1009.

The therapy and/or imaging transducers can be disposed within in the coupling assembly 1001 which can further include a coupling membrane 1014 and a membrane constraint 1016 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 1001 is supported by a mechanical support arm 1018 which can be load bearing in the x-y plane but allow for manual or automated z-axis adjustment. The mechanical support arm can be attached to the floor, the patient table, or the fluidics cart 1010. The mechanical support is designed and configured to conform and hold the coupling membrane 1014 in place against the patient's skin while still allowing movement of the therapy/imaging transducer relative to the patient and also relative to the coupling membrane 1014 with the robotic positioning arm 1008.

The fluidics cart 1010 can include additional features, including a fluid tank 1020, a cooling and degassing system, and a programmable control system. The fluidics cart is configured for external loading of the coupling membrane with automated control of fluidic sequences. Further details on the fluidics cart are provided below.

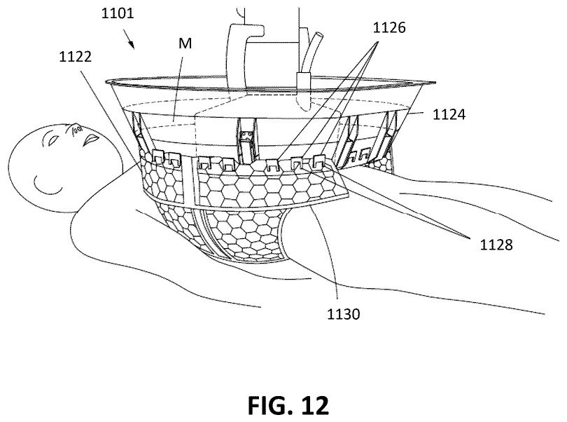

FIG. 12 illustrates one embodiment of a constraint device 1122 coupled to a coupling assembly 1101 having a frame body 1124 with a patient laying on a patient portion of the constraint device. Particularly, at least some of the attachment features 1126 of the coupling assembly are positioned through some of the openings 1028 of the peripheral portion 1130 of the constraint device 1122 to attach (i.e., hang, suspend, connect) the constraint device to the coupling assembly. The coupling membrane can be expanded as the coupling assembly 1101 is filled with an ultrasound coupling medium M for acoustic coupling to an ultrasound transducer array. In this example, the stiffness or flexibility of the membrane constraint relative to the stiffness or flexibility of the coupling membrane prevents overexpansion of the coupling membrane. In particular, the stiffness or flexibility or the membrane constraint is less flexible and has a lower % elongation compared to the coupling membrane.

The flared, angled, or curved edges of the peripheral portions of the constraint device allow for the constraint device to be attached at various points along the coupling assembly while also maintaining a close fit to the contours of the patient. Depending on the location of the target tissue, the patient may be positioned on his or her back, stomach, or side. The flared peripheral portions provide flexibility in how the constraint is attached to the coupling assembly, enabling a variety patient positions, treatment head locations, and coupling assembly locations.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the coupling assembly can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “coupling assembly” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the coupling assembly. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium. The fluidics system may include the ability to degas, chill, monitor, adjust, dispense/fill, and retrieve/drain coupling medium to/from the coupling frame/assembly.

The fluidics system may include an emergency high flow rate system for rapid filling and draining of the coupling medium from the coupling assembly. The fluidics system may be configured to fill the coupling assembly with fluid on demand, or with predetermined fill amounts (e.g., automatic fill of a present volume of fluid such as 1 L, 3 L 6 L, 9 L, etc.).

In some implementations, the fluidics system is configured to connect to or receive fluid from a fluid source such as tap water. The fluidics system can include a degas system or mechanism such as a degas membrane that can be configured to degas fluid as it flows from the fluid source into the fluid tank of the fluidics system. The degas system can be further configured to degas the fluid as it flows from the fluid tank to the coupling assembly. In some implementations, the fluid is degassed to a first degas threshold while the fluid tank is filled from the fluid source, and held at the first degas threshold. The fluidics system can then further degas the fluid as it is transferred from the fluid tank to the coupling assembly (e.g., to a second degas threshold).

In some embodiments, the fluidics system can be configured for a single use of the coupling medium, or alternatively, for re-use of the medium. In some embodiments, the fluidics system can implement positive air pressure or vacuum to carry out leak tests of the coupling assembly and membrane prior to filling with a coupling medium. Vacuum assist can also be used for removal of air from the coupling assembly during the filling process. The fluidics system can further include filters configured to prevent particulate contamination from reaching the coupling assembly.

The fluidics system may implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

The cart may be powered through standard electrical service/connectors, as well as with a battery to allow for portable or off-grid use. The battery may also provide emergency power. The cart may also comprise a nitrogen tank and/or air compressor (not shown) for allowing blow down of the main/drain tubing to enable ensuring they are maintained dry/clean (under a nitrogen blanket). In some examples, the cart may include various processors or electronic controllers configured for programming/monitoring/reporting water status and parameters. Parameters may include oxygen saturation, temperature, particulate debris, pH, mix ratio, flow rate, fill level, power level/battery level, etc., which can be detected in real-time by any number of sensors disposed within and around the system. The parameters may be read out on a UI screen on the fluidics cart, and/or may be displayed/controlled on the therapy system cart display (through software UI).

The degassing module may contain filters or degassing membranes configured to remove particulate/debris, a de-gas contactor and a vacuum or peristaltic pump to move fluid through the system. In some examples, filters may be 0.2 micron in pore size. The de-gas contactor may be able to pull down to parts per billion, with around 3 gallon per minute flow, and capable of removing dissolved O₂, CO₂ and N₂ gas. Vacuum pumps may include key features such as pure transfer and evacuation, high compatibility with vapors and condensation, chemical resistance, and gas tight (very low leakage). In some examples, vacuum pumps are cable of pulling down to 8 torr. In some embodiments, the degassing system can omit the pump and can rely on the water source flow rate (e.g., tap water flow rate) to move the fluid through the system.

The tubing/connectors/lines, plastic and/or metallic, are configured to allow fluid and air communication through the system and overall acoustic/patient coupling system. These may also contain various components such as valves (e.g., two way, three way, etc.).

The electronic and manual controls provide system and user-facing system controls over all the functions of the system, including but not limited to pump and de-gassing controls. The control systems may further comprise various sensors, in-line and onboard, for sensing temperature, pressure, flow rate, dissolved oxygen concentration, volume, etc.

The fluidics system and cart may also have various electrical connections for power including leveraging external power, and/or may comprise a battery/toroid for enabling a detethered fully mobile configuration. This allows the fluidics cart to be wheeled up to prepare/set up a histotripsy procedure, and then wheel away once all fluidics related work-flow steps are complete, so as to not require the fluidics cart to be patient side during treatment/therapy.

The fluidics cart architecture and design may also include handles, individual or central locking casters, a top work surface, embedded user display devices, connectivity (e.g., ethernet, etc.), and may be designed to allow further integration of the support arm in some embodiments. It may also be outfitted with long/extended tubing to support intra-imaging system filling/draining, if for example, use within a CT or MRI, is desirable, so as to not have the overall medium/water volume in close proximity to the scanner, and/or filling during set up is required to further assess image/body divergence pre/post filling.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A transducer array, comprising:
an array shell comprising a solid emitting surface and a rear surface, a plurality of wells formed in the rear surface;
a plurality of first matching layers individually disposed in the plurality of wells and contacting the proximal surface; and
a plurality of transducer elements disposed in the wells and contacting the plurality of first matching layers, the plurality of transducer elements being configured to transmit ultrasound energy through the plurality of first matching layers and through the solid emitting surface towards a common focal point.

2. The transducer array of claim 1, wherein the solid emitting surface forms a second matching layer for the plurality of transducer elements.

3. The transducer array of claim 1, wherein the solid emitting surface is concave.

4. The transducer array of claim 1, wherein the solid emitting surface has a contiguous smooth curvature.

5. The transducer array of claim 1, wherein the solid emitting surface has flat facets corresponding to each of the plurality of wells formed in the rear surface.

6. The transducer array of claim 1, wherein the plurality of wells have the same surface area.

7. The transducer array of claim 1, wherein the plurality of wells have varying shapes.

8. The transducer array of claim 6, wherein the plurality of wells have varying shapes.

9. The transducer array of claim 1, wherein a bottom surface of each of the plurality of wells is flat.

10. The transducer array of claim 1, wherein each of the plurality of transducer elements are sized and shaped to fill out all edges of a corresponding well of the plurality of wells.

11. The transducer array of claim 1, wherein the plurality of wells are formed by a machining process.

12. The transducer array of claim 11, wherein the array shell comprises a plastic material.

13. The transducer array of claim 1, wherein the plurality of first matching layers comprises a polymer composite material.

14. The transducer array of claim 13, wherein the polymer composite material includes glass or ceramic particles disposed therein.

15. The transducer array of claim 1, wherein the plurality of first matching layers has an acoustic impedance of about 5-8 Megarayl.

16. The transducer array of claim 1, wherein the plurality of first matching layers has a thickness of from about 0.5 mm to 1.5 mm.

17. The transducer array of claim 1, wherein each of the plurality of transducer elements includes an electrical connection interface.

18. The transducer array of claim 17, wherein each of the plurality of transducer elements includes a first notch or cutout configured to allow the electrical connection interface to receive one or more wires.

19. The transducer array of claim 18, wherein each of the plurality of first matching layers comprises a second notch or cutout configured to align with the electrical connection interface and first notch or cutout of a corresponding transducer element of the plurality of transducer elements.

20. The transducer array of claim 18, wherein each of the plurality of transducer elements comprises a second notch or cutout configured to be used as a guide for applying an adhesive or encapsulating layer between each of the plurality of transducer elements and the plurality of first matching layers.

21. The transducer array of claim 17, wherein the electrical connection interface is disposed in a corner of each of the plurality of transducer elements.

22. The transducer array of claim 1, wherein a bottom surface of each of the plurality of wells has curved edges.

23. The transducer array of claim 22, wherein the plurality of first matching layers have curved front edges to match the curved edges of the plurality of wells.

24. The transducer array of claim 1, wherein the plurality of transducer elements comprise a piezoelectric-polymer composite material.

25. The transducer array of claim 1, wherein the plurality of transducer elements comprise a solid piezoelectric material.

26. The transducer array of claim 1, wherein the plurality of transducer elements comprise a silicon material formed with microelectromechanical systems (MEMS) technology.

27. The transducer array of claim 1, further comprising an overmolded epoxy layer disposed on the solid emitting surface of the array shell.

28. The transducer array of claim 1, wherein the overmolded layer forms a second matching layer.

29. The transducer array of claim 28, wherein the overmolded layer comprises an epoxy or urethane.

30. The transducer array of claim 1, further comprising a central aperture disposed in the array shell configured to receive an ultrasound imaging probe.
